(19) United States
(12) Patent Application Publication
(10) Pub. No.: US 2022/0235350 A1
(43) Pub. Date: Jul. 28, 2022

(54) SYSTEM AND METHOD FOR PREPARING MRNA

(71) Applicant: ACADEMY OF MILITARY MEDICAL SCIENCES, Beijing (CN)

(72) Inventors: Shengqi WANG, Beijing (CN); Rui XIAO, Beijing (CN); Hongjuan WEI, Beijing (CN); Zhen RONG, Beijing (CN); Liyan LIU, Beijing (CN)

(21) Appl. No.: 17/627,747

(22) PCT Filed: Nov. 19, 2019
(86) PCT No.: PCT/CN2019/119385

§ 371 Date: Jan. 17, 2022

(30) Foreign Application Priority Data
Jul. 17, 2019 (CN) 201910648409.7

(51) Int. Cl. C12N 15/10; B01L 7/00

(52) U.S. Cl. CPC C12N 15/10; B01L 7/52; B01L 2400/0666; B01L 2300/1822; B01L 2300/1894; B01L 2200/141

(57) ABSTRACT

The present application belongs to the technical field of mRNA preparation, and discloses a system and a method for preparing mRNA, which can shorten a preparation period, and avoid degradation of an mRNA product due to the introduction of RNase by manual pipetting. The system for preparing mRNA comprises a PCR amplification device configured to amplify DNA; a plurality of raw reagent tubes, each of which is configured to provide a single reagent; and a reaction device configured to allow reagents to react. Specifically, the reaction device comprises a test tube rack configured to support reaction tubes, a semiconductor chilling plate connected to the test tube rack, a fin type radiator disposed under the test tube rack, and a cooling fan disposed under the fin type radiator; wherein the raw reagent tubes are connected to the reaction tubes via a solenoid valve module and a peristaltic pump module, and the solenoid valve module and the peristaltic pump module are connected with a control device and are controlled by an upper computer control program.

### CROSS-REFERENCE TO RELATED APPLICATIONS

The present disclosure claims priority to Chinese Patent Application No. 201910648409.7, entitled “System and method for preparing mRNA” and filed with Chinese Patent Office on Jul. 17, 2019.

### TECHNICAL FIELD

The present disclosure belongs to the technical field of mRNA preparation, and particularly relates to a system and a method for preparing mRNA.

### BACKGROUND ART

An mRNA vaccine is a nucleic acid vaccine with immunity, safety and flexibility, and compared with a traditional vaccine, the mRNA vaccine has characteristics of high safety, high research and development speed, low research and development cost, easy mass production and on-demand preparation.

During the preparation of the mRNA vaccine, most of reaction raw materials in raw material tubes and reaction reagents in reaction tubes contain enzyme, and are required to be stored in an environment at 4° C. in addition, the existing preparation of the mRNA vaccine may be completed by a plurality of apparatuses and more manpower, resulting in a long preparation period. Furthermore, in a traditional method for preparing mRNA, a liquid addition and transferring (pipetting operation) is manually performed using a pipetting gun, such that RNase is easy to be introduced, resulting in degradation of the mRNA and a relatively low yield.

Therefore, how to provide a system and a method for preparing mRNA to shorten the preparation period and avoid contamination caused by the manual pipetting operation becomes an urgent technical problem for those skilled in the art.

### SUMMARY

An object of the present disclosure is, for example, to provide a system and a method for preparing mRNA, which can shorten a preparation period, and avoid degradation of mRNA due to the introduction of RNase by manual pipetting.

Embodiments of the present disclosure are implemented as follows.

The system for preparing mRNA includes a PCR amplification device configured to amplify DNA; a plurality of raw reagent tubes, each of which is configured to provide a single reagent; and at least one reaction device configured to allow reagents to react, wherein the reaction device comprises a test tube rack configured to support a reaction tube, a semiconductor chilling plate connected to the test tube rack, a fin type radiator disposed under the test tube rack, and a cooling fan disposed under the fin type radiator; the raw reagent tubes are connected to the reaction tubes via a solenoid valve assembly and a peristaltic pump assembly, and the solenoid valve assembly and the peristaltic pump assembly are connected with a control device and are controlled by an upper computer control program.

Optionally, the PCR amplification device includes at least one PCR heating column each connected to both the solenoid valve assembly and the peristaltic pump assembly, and the PCR heating column is configured to amplify template DNA.

Optionally, the raw reagent tubes include: a first reagent tube, a second reagent tube, a third reagent tube and a fourth reagent tube; the first reagent tube is configured to contain a capture reagent; the second reagent tube is configured to contain a transcription reagent; the third reagent tube is configured to contain an elution reagent; and the fourth reagent tube is configured to contain a washing reagent.

Optionally, the fin type radiator is any one of a wrapped fin type radiator, a serial fin type radiator, a welded fin type radiator and an extruded fin type radiator.

Optionally, the reaction device further includes a housing covering outer sides of the test tube rack and the semiconductor chilling plate and butt-jointed with the fin type radiator, wherein one end of the reaction tube extends out of the housing.

Optionally, the housing and the fin type radiator together define a cavity configured to accommodate the test tube rack.

Optionally, the reaction device is provided with a plunger pump assembly, and the plunger pump assembly is in communication with the reaction tube.

Optionally, any one of a single plunger pump, a horizontal plunger pump, an axial plunger pump and a radial plunger pump is adopted in the plunger pump assembly.

Optionally, plural reaction devices are provided, the system for preparing mRNA further includes a magnetic attraction assembly, and two adjacent reaction devices of the plural reaction devices are connected by one common magnetic attraction assembly.

Optionally, four reaction devices are provided.

Optionally, the magnetic attraction assembly includes a motor and a magnet connected with the motor, and the motor is configured to drive the magnet to reciprocate between the adjacent reaction devices.

Optionally, in the present disclosure, the system for preparing mRNA further includes a waste liquid collection bottle in communication with the reaction tube and configured to collect waste liquid in the reaction tube.

Optionally, the waste liquid collection bottle is in communication with the reaction tube sequentially through the peristaltic pump assembly and the solenoid valve assembly.

Optionally, the system for preparing mRNA further includes a base, and both the reaction device and the PCR amplification device are connected to the base.

Optionally, the system for preparing mRNA further includes a support frame connected to the base, an accommodating space is provided between the support frame and the base, and the PCR amplification device is located in the accommodating space; and the raw reagent tube, the solenoid valve assembly and the peristaltic pump assembly are all connected to the support frame.

Optionally, the support frame includes a top plate, a first side plate and a second side plate, the first side plate and the second side plate are both connected to the top plate, and the first side plate and the second side plate are both connected to the base; the raw reagent tube, the solenoid valve assembly and the peristaltic pump assembly are all connected to the top plate; and the control device is connected to the second side plate.

The embodiments of the present disclosure have the following beneficial effects, for example.

The semiconductor chilling plate in the reaction device is configured to chill or heat the test tube rack, the reaction tube is inserted in the test tube rack, and a temperature of the reaction tube is controlled by the cooling fan and the fin type radiator in conjunction with the semiconductor chilling plate, thus ensuring that a preservation temperature of the reagent in the reaction tube is 4° C., and enabling it to quickly reach a temperature required by the reaction of the reagent; in addition, the raw reagent tube is connected with the reaction device in the reaction tube by the solenoid valve assembly and the peristaltic pump assembly, and the PCR amplification device is connected with both the solenoid valve assembly and the peristaltic pump assembly, such that flow of liquid may be well controlled by controlling the solenoid valve assembly and the peristaltic pump assembly to be opened or closed, thus effectively avoiding the degradation of the mRNA due to the introduction of the RNase by manual pipetting, and effectively increasing a yield of the mRNA; the DNA is amplified by the PCR amplification device in the flowing process of the liquid, thus achieving a good amplification effect; meanwhile, a plurality of components are integrated in the system for preparing mRNA, such that an instrument volume is reduced as much as possible, an experimenter may observe the whole experiment process conveniently, and the preparation period is short.

Meanwhile, the adoption of the semiconductor chilling plate has advantages that the semiconductor chilling plate may be used in a small narrow space, has high reliability and rapid chilling and is free of refrigerant pollution, and no additional cold source is required; by using a Peltier effect of a semiconductor material, when a direct current passes through a galvanic couple formed by two different semiconductor materials in series, heat may be rapidly absorbed and released at two ends of the galvanic couple respectively, thereby achieving a rapid chilling purpose and effectively shortening the preparation period.

Another object of the present disclosure is to provide a method for preparing mRNA, which may conveniently and rapidly prepare an mRNA vaccine.

An embodiment of the present disclosure is implemented as follows.

The method for preparing mRNA, in which the system for preparing mRNA according to any one of the above descriptions is used for production and preparation, includes the following steps: amplifying template DNA using the PCR amplification device; performing a biotin affinity test by the solenoid valve assembly and the peristaltic pump assembly; performing capping modification by the solenoid valve assembly and the peristaltic pump assembly; performing tailing transcription by the solenoid valve assembly and the peristaltic pump assembly; and purifying an mRNA product by the solenoid valve assembly and the peristaltic pump assembly.

The embodiment of the present disclosure has the following beneficial effects, for example.

Flow of liquid may be well controlled by controlling the solenoid valve assembly and the peristaltic pump assembly to be opened or closed, thus effectively avoiding the degradation of the mRNA due to the introduction of the RNase by manual pipetting, effectively increasing a yield of the mRNA, and conveniently and rapidly preparing the mRNA vaccine.

Here, it should be additionally noted that mRNA, also called messenger RNA, is transcribed from one strand of DNA as a template, usually includes a 5′ untranslated region (UTR), an open reading frame (ORF), a 3′ UTR, and other structures, and is single-stranded ribonucleic acid which carries genetic information and may direct protein synthesis.

Furthermore, DNA is also called deoxyribonucleic acid which is an organic compound having a complicated molecular structure. DNA exists in a cell nucleus as a component of a chromosome. DNA has a function of storing genetic information. A DNA molecule is large and composed of nucleotide. A nitrogenous base of the nucleotide is adenine, guanine, cytosine and thymine; pentose is deoxyribose. In 1953, James Dewey Watson in the United states as well as Crick and Wilkins in the United Kingdom described the structure of DNA: a pair of polynucleotide strands is coiled around a common central axis. A sugar-phosphate strand is located at the exterior of the helical structure, and the base faces inwards. The two polynucleotide strands are linked by hydrogen bonds between the bases to form a fairly stable combination.

### BRIEF DESCRIPTION OF DRAWINGS

To describe the technical solutions in the embodiments of the present disclosure more clearly, the following briefly describes the accompanying drawings required in the embodiments. It should be understood that the following accompanying drawings show merely some embodiments of the present disclosure and therefore should not be considered as limiting the scope, and a person of ordinary skill in the art may still derive other related drawings from these accompanying drawings without creative efforts.

FIG. 1 is a schematic structural diagram of a system for preparing mRNA according to the present disclosure;

FIG. 2 is a schematic exploded structural diagram of a reaction device in the system for preparing mRNA according to the present disclosure;

FIG. 3 is a schematic flow chart of a method for preparing mRNA according to the present disclosure; and

FIG. 4 is a schematic diagram of a system for automatically and rapidly synthesizing an mRNA vaccine according to the present disclosure.

In the drawings:

1—reaction device; 11—reaction tube; 12—test tube rack; 13—semiconductor chilling plate; 14—fin type radiator; 15—cooling fan; 16—housing; 2—plunger pump assembly; 3—magnetic attraction assembly; 5—raw reagent tube; 6—solenoid valve assembly; 7—peristaltic pump assembly; 8—control device; 9—waste liquid collection bottle; 10—PCR heating column; 100—base; 200—support frame; 210—top plate; 220—first side plate; 230—second side plate; 300—socket; 400—accommodating space; 500—PCR amplification device.

### DETAILED DESCRIPTION

To make the objectives, technical solutions and advantages of the embodiments of the present disclosure clearer, the technical solutions in the embodiments of the present disclosure are clearly and completely described with reference to the accompanying drawings in the embodiments of the present disclosure, and apparently, the described embodiments are not all but a part of the embodiments of the present disclosure. Generally, the components of the embodiments of the present disclosure described and illustrated in the drawings herein may be arranged and designed in a variety of different configurations.

Accordingly, the following detailed description of the embodiments of the present disclosure provided in the drawings is not intended to limit the scope of protection of the present disclosure, but only represents selected embodiments of the present disclosure. All other embodiments obtained by a person of ordinary skill in the art based on the embodiments of the present disclosure without creative efforts shall fall within the protection scope of the present disclosure.

It should be noted that similar reference signs and letters denote similar items in the following drawings. Therefore, once a certain item is defined in one figure, it does not need to be further defined and explained in the subsequent figures.

In descriptions of the present disclosure, it should be noted that directions or positional relationships indicated by terms “center”, “upper”, “lower”, “left”, “right”, “vertical”, “horizontal”, “inner”, “outer”, etc. are based on orientations or positional relationships shown in the accompanying drawings, or orientations or positional relationships of conventional placement of the product according to the present invention in use, and they are used only for describing the present disclosure and for simplifying the description, but do not indicate or imply that an indicated device or element must have a specific orientation or be constructed and operated in a specific orientation. Therefore, it cannot be understood as a limitation on the present disclosure. In addition, the terms such as “first”, “second”, “third”, or the like, are only used for distinguishing descriptions and are not intended to indicate or imply importance in relativity.

In addition, the terms of “horizontal”, “vertical”, and “overhung” and so on do not represent that it requires that the component is absolutely horizontal or overhung but can be slightly tilted. For example, “horizontal” only means that the direction is more horizontal than “vertical”, but does not mean that the structure has to be horizontal completely, instead, it can be slightly tilted.

In the description of the present disclosure, it still should be noted that unless clearly specified or defined otherwise, the terms “provided”, “mounted”, “connected”, and “coupled” and the like should be understood broadly, and may be, for example, fixed connections, detachable connections, or integral connections; may also be mechanical connections, or may be also electrical connections; may also be direct connections or indirect connections via an intermediary medium; or may also be inner communications between two elements. The specific meanings of the above terms in the present disclosure can be understood by those skilled in the art according to specific situations.

Some embodiments of the present disclosure will be described in detail below with reference to the accompanying drawings. The embodiments described below and features therein may be combined with each other without conflicts.

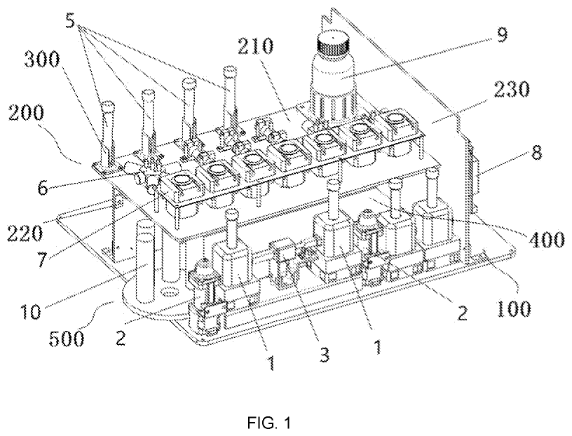

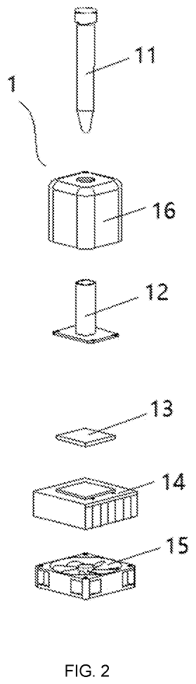

Referring to FIGS. 1 to 2, the present disclosure provides a system for preparing mRNA, including a PCR amplification device 500, raw reagent tube(s) 5 and reaction devices 1; the PCR amplification device 500 is configured to amplify DNA; the number of the raw reagent tubes 5 is set as required; for example, the number of the raw reagent tubes 5 is plural, and each raw reagent tube 5 is configured to provide a single reagent; the reaction device 1 is configured to allow reagents to react. Optionally, the reaction device 1 includes a reaction tube 11, a test tube rack 12, a semiconductor chilling plate 13, a fin type radiator 14 and a cooling fan 15. The test tube rack is configured to support the reaction tube 11, the test tube rack 12 is connected with the semiconductor chilling plate 13, the fin type radiator 14 is disposed under the test tube rack 12, and the cooling fan 15 is disposed under the fin type radiator 14; here, the raw reagent tubes 5 are connected to the reaction tubes 11 via a solenoid valve assembly 6 and a peristaltic pump assembly 7, and both the solenoid valve assembly 6 and the peristaltic pump assembly 7 are connected with a control device 8 and are controlled by an upper computer control program.

Optionally, the system for preparing mRNA further includes a waste liquid collection bottle 9, wherein the waste liquid collection bottle 9 is in communication with the reaction tubes 11 sequentially through the peristaltic pump assembly 7 and the solenoid valve assembly 6 and configured to collect waste liquid in the reaction tubes 11. The arrangement of the waste liquid collection bottle 9 not only facilitates collection of the waste liquid generated in the reaction tubes 11, but also may effectively reduce pollution of the waste liquid to an environment.

Optionally, the system for preparing mRNA further includes a base 100 and a support frame 200 disposed on the base 100, the base 100 may be of a flat plate structure, the support frame 200 is disposed on a plate surface of the base 100, the support frame 200 and the base 100 may be fixedly connected to each other by screws, bolts, rivets, welding, or the like, and an accommodating space 400 is formed between the support frame 200 and the base 100. The reaction device 1 is provided on the base 100. Optionally, the cooling fan 15 of the reaction device 1 is connected to the base 100, and the reaction device 15 may be connected to the base 100 by screws, bolts, or rivets.

Optionally, the support frame 200 includes a top plate 210, a first side plate 220 and a second side plate 230, the first side plate 220 and the second side plate 230 are both connected to the top plate 210, the first side plate 220 and the second side plate 230 are both connected to a bottom plate, the top plate 210 is parallel to the bottom plate, and the top plate 210 and the bottom plate have a spacing therebetween. Optionally, the first side plate 220 is perpendicular to the second side plate 230. The top plate 2105 is provided with sockets 300 where the raw reagent tubes 5 are inserted, the number of the sockets 300 is equal to the number of the raw reagent tubes 5, and the sockets and the raw reagent tubes 5 in one-to-one insertion fit. The solenoid valve assembly 6, the peristaltic pump assembly 7 and the waste liquid collection bottle 9 are all provided on the top plate 210, and the control device 8 is provided on the second side plate 230.

Referring to FIG. 1, in the present disclosure, the PCR amplification device 500 may include a PCR heating column 10 connected with both the solenoid valve assembly 6 and the peristaltic pump assembly 7, such that the DNA may be amplified by the PCR amplification device 500. In other words, the PCR heating column 10 is heated to exchange heat with the reagent flowing in the solenoid valve assembly 6 and the peristaltic pump assembly 7 to heat the reagent, thereby amplifying the DNA.

Here, it should be additionally noted that a polymerase chain reaction is abbreviated as PCR, the PCR is a method for synthesizing a specific DNA fragment in vitro by enzyme, wherein reactions, such as high-temperature denaturation, low-temperature annealing (renaturation), suitable-temperature extension, or the like, form a period and are performed circularly, such that the target DNA may be amplified rapidly, and the PCR has characteristics of high specificity, high sensitivity, simple and convenient operation, time saving, or the like.

In the present disclosure, the raw reagent tubes 5 may include: a first reagent tube, a second reagent tube, a third reagent tube and a fourth reagent tube; optionally, the first reagent tube may be configured to contain a capture reagent; the second reagent tube may be configured to contain a transcription reagent; the third reagent tube may be configured to contain an elution reagent; and the fourth reagent tube may be configured to contain a washing reagent.

It should be noted that the number of the raw reagent tubes 5 may be not limited to four; for example, the number of the raw reagent tubes 5 may be 3, 5, or the like.

In the present disclosure, the fin type radiator 4 may be any one of a wrapped fin type radiator, a serial fin type radiator, a welded fin type radiator and an extruded fin type radiator.

Referring to FIGS. 1 to 2, in the present disclosure, the reaction device 1 may further include a housing 16, wherein the housing 16 covers outer sides of the test tube rack 12 and the semiconductor chilling plate 13, and is butt-jointed with the fin type radiator 14. The housing 16 and the fin type radiator 14 together define a cavity, a temperature in the cavity is stable, the test tube rack 12 is located in the cavity, the reaction tube 11 is inserted into the test tube rack 12, and one end of the reaction tube extends out of the housing 6, thus facilitating maintenance of the temperature of the reaction tube 11 and the preparation of the mRNA.

Referring to FIGS. 1 to 2, in the present disclosure, the reaction device 1 may be provided with a plunger pump assembly 2, the plunger pump assembly 2 is disposed on the base 100, and the plunger pump assembly 2 is in communication with the reaction tube 11, such that traditional oscillation mixing is replaced by the plunger pump assembly 2, and thus, a volume and complexity of the instrument are reduced effectively, thereby reducing a cost.

In the present disclosure, any one of a single plunger pump, a horizontal plunger pump, an axial plunger pump and a radial plunger pump may be adopted in the plunger pump assembly 2.

Referring to FIGS. 1 to 2, in the present disclosure, four reaction devices 1 may be provided, two of the reaction devices are configured to achieve a magnetic attraction function, and the two reaction devices 1 required to achieve the magnetic attraction function may be connected to each other by a common magnetic attraction assembly 3.

Referring to FIGS. 1 to 2, in the present disclosure, the magnetic attraction assembly 3 may include a motor and a magnet connected with the motor, the motor is provided on the base 100, and the motor rotates to drive the magnet to move in parallel; that is, the magnet may slide, under driving of the motor, back and forth between the two reaction devices 1 required to achieve the magnetic attraction function. The two reaction devices 1 share one magnet, thus effectively reducing the volume of the instrument, and reducing the cost.

The system for preparing mRNA according to the present disclosure has the following beneficial effects, for example.

The semiconductor chilling plate 13 in the reaction device 1 is configured to chill or heat the test tube rack 12, the reaction tube 11 is inserted in the test tube rack 12, and the temperature of the reaction tube 11 is controlled by the cooling fan 15 and the fin type radiator 14 in conjunction with the semiconductor chilling plate 13, thus ensuring that the temperature of the reaction tube 11 is stabilized at a required temperature; in addition, in the PCR amplification device 500, each raw reagent tube 5 and the corresponding reaction device 1 are connected by the solenoid valve assembly 6 and the peristaltic pump assembly 7, and the PCR amplification device 500 is connected with both the solenoid valve assembly 6 and the peristaltic pump assembly 7, such that flow of liquid may be well controlled by controlling the solenoid valve assembly 6 and the peristaltic pump assembly 7 to be opened or closed, thus effectively avoiding degradation of the mRNA due to the introduction of the RNase by manual pipetting, and effectively increasing a yield of the mRNA; the DNA is amplified by the PCR amplification device 500 in the flowing process of the liquid, thus achieving a good amplification effect; meanwhile, a plurality of components are integrated in the system for preparing mRNA, such that the volume of the instrument is reduced as much as possible, an experimenter may observe the whole experiment process conveniently, and a preparation period is short.

Meanwhile, the adoption of the semiconductor chilling plate 13 has advantages that the semiconductor chilling plate may be applied in a small narrow space, has high reliability and rapid chilling and is free of refrigerant pollution, and no additional cold source is required; by using a Peltier effect of a semiconductor material, when a direct current passes through a galvanic couple formed by two different semiconductor materials in series, heat may be rapidly absorbed and released at two ends of the galvanic couple respectively, thereby achieving a rapid chilling purpose and effectively shortening the preparation period.

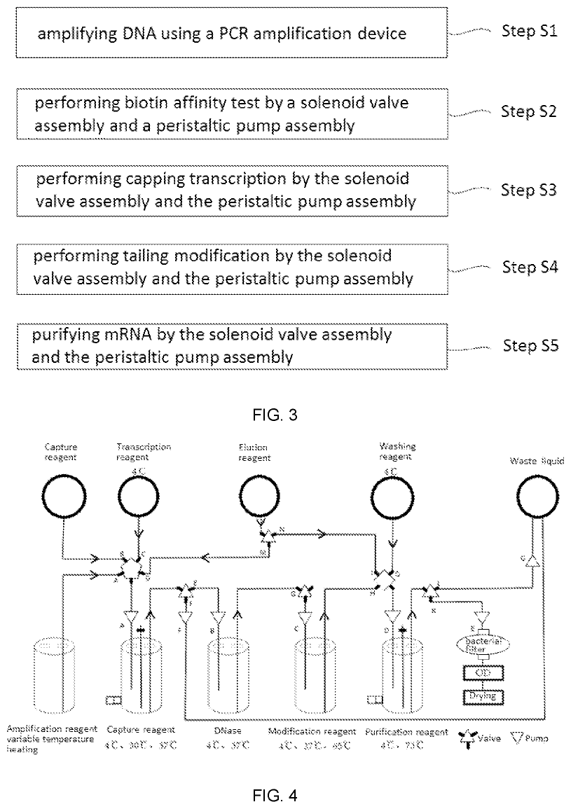

Referring to FIG. 3, the present disclosure provides a method for preparing mRNA, in which the system for preparing mRNA according to the present disclosure is used for production and preparation of the mRNA; the method includes the following steps:

step S1: amplifying template DNA using the PCR amplification device; step S2: performing a biotin affinity test in the at least one reaction device 1 by the corresponding solenoid valve assembly 6 and peristaltic pump assembly 7; step S3: performing capping transcription in the at least one reaction device 1 by the corresponding solenoid valve assembly 6 and peristaltic pump assembly 7; step S4: performing tailing modification in the at least one reaction device 1 by the corresponding solenoid valve assembly 6 and peristaltic pump assembly 7; and step S5: purifying an mRNA product in the at least one reaction device 1 by the corresponding solenoid valve assembly 6 and peristaltic pump assembly 7.

In an actual operation, as shown in FIG. 4, specific steps may include:

step S1: amplifying 0.5 ml using the PCR amplification device:

95° C. kept for 5 min—[95° C. kept for 40 s—58° C. kept for 45 s—72° C. kept for 1 min] (30 cycles)—72° C. kept for 10 min;

step S21: amplification reagent 0.5 ml—only opening valve A and pump A—capture reagent 2.5 ml:

under a condition of a temperature of 30° C., oscillation and maintenance for 45 min—1 h [magnet adsorption], supernatant 3 ml—only opening valve F and pump F—waste liquid collection bottle (magnet moved away);

step S22: capture reagent 2.5 ml—only opening valve B and pump A—capture reagent:

oscillation [magnet adsorption], supernatant 2.5 ml—only opening valve F and pump F—waste liquid collection bottle (magnet moved away) (repeated twice);

step S23: eluent 2.5 ml—only opening valve M, valve D and pump A—capture reagent:

oscillation [magnet adsorption], supernatant 2.5 ml—only opening valve F and pump F—waste liquid collection bottle (magnet moved away);

step S31: transcription reagent 0.5 ml—only opening valve C and pump A—capture reagent:

under a condition of a temperature of 37° C., oscillation and maintenance for 4 h [magnet adsorption];

step S32: capture reagent (post-transcription), then supernatant 0.5 ml—only opening valve E and pump B—DNase 30 μl:

under a condition of a temperature of 37° C., oscillation and maintenance for 15 min [magnet adsorption];

step S4: adding DNase and 0.53 ml of supernatant—only opening valve G and pump C—modification reagent 0.54 ml:

maintaining 37° C. for 1 h, rapidly heating to 65° C. , and maintaining the temperature for 5 min;

step S51: adding a modification reagent and 1.07 ml of supernatant—only opening valve H and pump D—purification reagent 1 ml:

under a condition of a room temperature, oscillation and maintenance for 5-10 min [magnet adsorption];

step S52: adding a purification reagent and 2.07 ml of supernatant—only opening valve L and pump G-waste liquid collection bottle (magnet moved away);

step S53: washing liquid 2 ml—only opening valve J and pump D—purification reagent:

oscillation [magnet adsorption], supernatant 2 ml—only opening valve L and pump G—waste liquid collection bottle (magnet moved away) (repeated twice);

step S54: eluent 0.2 ml—only opening valve N, valve I and pump D—purification reagent:

under a condition of a temperature of 73° C., maintaining oscillation for 2 min [magnet adsorption], and supernatant 0.2 ml—only opening valve K and pump E—product (magnet move away) (repeated for three times).

MRNA vaccine—for 10 persons

Raw reagent tube; reaction tube

Capture reagent: 0.72M binding solution, room temperature, 5 ml; amplification reagent: PCR 0.5 ml

Transcription reagent: capping transcription system, 4° C., 0.5 ml; capture reagent: 2.5 ml of streptavidin magnetic beads resuspended after 3 times of washing using 1 ml of 0.72M binding solution

Eluent: 0.1% DEPC water, room temperature, 3.5 ml; DNase: 30 μl

Washing liquid: Washing, 4° C. , 4 ml; modification reagent: tailing transcription system 0.54 ml

Purification reagent: 1 ml of Oligod (T) magnetic beads resuspended in 100 μl after one time of washing using 100 μl of Binding.

The present disclosure has the following beneficial effects. Flow of liquid may be well controlled by controlling the solenoid valve assembly 6 and the peristaltic pump assembly 7 to be opened or closed, thus effectively avoiding the degradation of the mRNA due to the introduction of the RNase by manual pipetting, effectively increasing a yield of the mRNA, and conveniently and rapidly preparing the mRNA vaccine.

It should be additionally noted here that the DNase (i.e., deoxyribonuclease) is endonuclease which may digest single-stranded or double-stranded DNA to generate single deoxynucleotide or single-stranded or double-stranded oligodeoxynucleotide. The RNase is enzyme acting only on RNA, widely exists in the environment, and has quite good stability and no sequence specificity, such that unprotected RNA is easily degraded by the RNase.

The above description is only preferred embodiments of the present disclosure and is not configured to limit the present disclosure, and various modifications and changes may be made to the present disclosure by those skilled in the art. Any modification, equivalent replacement, or improvement made within the spirit and principle of the present disclosure shall be included in the protection scope of the present disclosure.

### INDUSTRIAL APPLICABILITY

In conclusion, the present disclosure provides the system and the method for preparing mRNA, which achieve the high yield of the mRNA.

1. A system for preparing mRNA, comprising:
a PCR amplification device, configured to amplify DNA;
a plurality of raw reagent tubes, each of which is configured to provide a single reagent; and
at least one reaction device, configured to allow reagents to react,
wherein the at least one reaction device each comprises a test tube rack configured to support a reaction tube, a semiconductor chilling plate connected to the test tube rack, a fin type radiator disposed under the test tube rack, and a cooling fan disposed under the fin type radiator; and
the raw reagent tube is connected to the reaction tube via a solenoid valve assembly and a peristaltic pump assembly, and the solenoid valve assembly and the peristaltic pump assembly are connected with a control device and are configured to be controlled by an upper computer control program.

2. The system for preparing mRNA according to claim 1, wherein the PCR amplification device comprises at least one PCR heating column connected to both the solenoid valve assembly and the peristaltic pump assembly, and the at least one PCR heating column is configured to amplify template DNA.

3. The system for preparing mRNA according to claim 1, wherein the raw reagent tubes comprise: a first reagent tube, a second reagent tube, a third reagent tube and a fourth reagent tube;
the first reagent tube is configured to contain a capture reagent;
the second reagent tube is configured to contain a transcription reagent;
the third reagent tube is configured to contain an elution reagent; and
the fourth reagent tube is configured to contain a washing reagent.

4. The system for preparing mRNA according to claim 1, wherein the fin type radiator is any one of a wrapped fin type radiator, a serial fin type radiator, a welded fin type radiator and an extruded fin type radiator.

5. The system for preparing mRNA according to claim 4, wherein the reaction device further comprises a housing, wherein the housing covers outer sides of the test tube rack and the semiconductor chilling plate and is connected with the fin type radiator, wherein one end of the reaction tube extends out of the housing.

6. The system for preparing mRNA according to claim 4, wherein the housing and the fin type radiator together define a cavity configured to accommodate the test tube rack.

7. The system for preparing mRNA according to claim 1, wherein the reaction device is provided with a plunger pump assembly, and the plunger pump assembly is in communication with the reaction tube.

8. The system for preparing mRNA according to claim 7, wherein any one of a single plunger pump, a horizontal plunger pump, an axial plunger pump and a radial plunger pump is adopted in the plunger pump assembly.

9. The system for preparing mRNA according to claim 1, wherein plural reaction devices are provided, the system for preparing mRNA further comprises a magnetic attraction assembly, and two adjacent reaction devices of the plural reaction devices are connected by one common magnetic attraction assembly.

10. The system for preparing mRNA according to claim 9, wherein four reaction devices are provided.

11. The system for preparing mRNA according to claim 9, wherein the magnetic attraction assembly comprises a motor and a magnet connected with the motor, and the motor is configured to drive the magnet to reciprocate between the adjacent reaction devices.

12. The system for preparing mRNA according to claim 11, further comprising a waste liquid collection bottle in communication with the reaction tubes and configured to collect waste liquid in the reaction tubes.

13. The system for preparing mRNA according to claim 12, wherein the waste liquid collection bottle is in communication with each of the reaction tubes sequentially through the peristaltic pump assembly and the solenoid valve assembly.

14. The system for preparing mRNA according to claim 13, further comprising a base, wherein both the reaction device and the PCR amplification device are connected to the base.

15. The system for preparing mRNA according to claim 14, further comprising a support frame connected to the base, wherein an accommodating space is provided between the support frame and the base, and the PCR amplification device is located in the accommodating space; and the raw reagent tube, the solenoid valve assembly and the peristaltic pump assembly are all connected to the support frame.

16. The system for preparing mRNA according to claim 15, wherein the support frame comprises a top plate, a first side plate and a second side plate, the first side plate and the second side plate are both connected to the top plate, and the first side plate and the second side plate are both connected to the base; the raw reagent tube, the solenoid valve assembly and the peristaltic pump assembly are all connected to the top plate; and the control device is connected to the second side plate.

17. A method for preparing mRNA, in which the system for preparing mRNA according to claim 1 is used for production and preparation, the method comprising following steps:
amplifying template DNA using the PCR amplification device;
performing a biotin affinity test by the solenoid valve assembly and the peristaltic pump assembly;
performing capping transcription by the solenoid valve assembly and the peristaltic pump assembly;
performing tailing modification by the solenoid valve assembly and the peristaltic pump assembly; and
purifying an mRNA product by the solenoid valve assembly and the peristaltic pump assembly.

18. The system for preparing mRNA according to claim 6, wherein the reaction drive is provided with a plunger pump assembly, and the plunger pump assembly is in communication with the reaction tube.

19. The system for preparing mRNA according to claim 8, wherein plural reaction devices are provided, the system for preparing mRNA further comprises a magnetic attraction assembly, and two adjacent reaction devices of the plural reaction devices are connected by one common magnetic attraction assembly.

20. The system for preparing mRNA according to claim 10, wherein the magnetic attraction assembly comprises a motor and a magnet connected with the motor, and the motor is configured to drive the magnet to reciprocate between the adjacent reaction devices.
